(19) United States
(12) Patent Application Publication
(10) Pub. No.: US 2003/0211453 A1
(43) Pub. Date: Nov. 13, 2003

(54) Fertility associated antigens and method of use thereof

(76) Inventors: Huanmin Zhang, Okemos, MI (US); Roy L Ax, Tucson, AZ (US)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/240,721

(22) PCT Filed: Apr. 9, 2001
(86) PCT No.: PCT/US01/10802

(51) Int. Cl. A01N 1/02; C07H 21/04; C12P 21/02; C12N 1/21; C12N 5/06; C07K 14/47

(52) U.S. Cl. 435/2; 435/69.3; 435/252.3; 435/320.1; 435/325; 536/23.5; 530/350

(57) ABSTRACT

The present invention relates to a gene encoding a fertility associated antigen methods of employing the fertility associated antigen to stabilize sperm and/or increase the fertility of a male and method of assessing, the fertility and/or reproductive fitness of a male

The present invention claims priority to U.S. Provisional Application No. 60/195,225 filed Apr. 7, 2000, the entire contents of which are incorporated herein by reference.

### BACKGROUND OF THE INVENTION

1. Field of the Invention

The present invention relates to a fertility-associated antigen (FAA) protein its complementary deoxynucleotide sequence, which encodes the FAA and identified single nucleotide polymorphisms (SNPs) of the human FAA gene. The invention also provides fundamental data for developing methods of predicting which candidates within a male mammal population are likely to be more fertile breeders, or to be facing difficulty in fathering offspring, by assaying the SNPs of their FAA gene or their FAA seminal protein, which is secreted by sex glands.

2. Discussion of the Background

It has been well documented that seminal fluid is a complex mixture consisting of secretions of the male accessory organs of reproduction: seminal vesicles (V. G.), prostate (P. G.), and bulbourethral glands (B. G.; Shivaji et al., 1990). Of the seminal fluid constituents, some have been shown to inhibit (Davis, 1976; Lenz et al., 1982) and others to stimulate (Florman and First, 1988; Miller et al., 1990) sperm capacitation in vitro.

Seminal components that stimulate capacitation include a family of heparin-binding proteins (HBP) that bind to sperm at ejaculation and convey heparin-induced capacitation (Miller, 1990). A murine monoclonal antibody (mAb), Ml, generated by immunization with purified HBP, recognized three distinct proteins in immunoblots of bovine sperm extracts (Bellin et al., 1996, 1998). One of the three HBPs was apparent to be a single 31-kDa mass (FIG. 1), and was described as fertility-associated antigen (FAA; Bellin et al., 1998).

Presence or absence of FAA on sperm relates to fertility demonstrated by data from breeding cattle at a ranch with bulls and artificial insemination (Table 1 and Table 2).

TABLE 1
FAA and Bull Fertility

Natural Service Data*

| | No. | No. | No. Cows | |
|---|---|---|---|---|
| Sperm | Bulls | Cows Bred | Pregnant | Pregnant % |
| FAA Positive | 242 | 5,317 | 4,497 | 85%^{a} |
| FAA Negative | 192 | 3,881 | 2,572 | 66%^{b} |
| Total | 434 | 9,198 | 7,069 | 19% Diff. |

*Multiple pastures
1 Bull for every 25 cows
^{a} vs ^{b}:P1 < 0.01

TABLE 2
FAA and Bull Fertility

Artificial Insemination Data*

| | No. | No. | No. Cows | |
|---|---|---|---|---|
| Sperm | Bulls | Cows Bred | Pregnant | Pregnant % |
| FAA Positive | 18 | 550 | 341 | 62%^{a} |
| FAA Negative | 7 | 315 | 144 | 45.7%^{b} |
| Total | 25 | 865 | 485 | 16.3% Diff. |

*Pregnancy rates to first AI service
^{a} vs ^{b}: P < 0.01

Male fertility is a significant problem in breeding programs for agricultural animals and for those human couples attempting to conceive a child. Attempts at either predicting the fertilization capacity of a male and/or subsequent treatment of the sperm to increase sperm function have proven to be largely unsuccessful.

Accordingly, there is a great need for both suitable means for first diagnosing the fertility of a male and increasing the sperm function/capacitation of the male with suitable treatments.

These and other problems are addressed by the present Inventor's discovery and characterization of the FAA from bovine and human disclosed herein.

### SUMMARY OF THE INVENTION

Accordingly, one object of the present invention is the isolated polynucleotides which are shown in either of SEQ ID NO: 1, 2, 3, 4, or 5. Particularly, the polynucleotides which encode the polypeptides having the amino acid sequences in SEQ ID NO: 6, 7, 8, 9, or 10, and more particularly, those proteins which are a fertility associate antigen.

Other preferred embodiments of the present invention is methods for increasing the stability and/or capacitation of a sperm cell by administering FAA to the sperm cell in an amount sufficient to increase the stability and/or capacitation of said sperm can aThe method can be performed in vitro or in vivo.

Other preferred embodiments of the present invention is methods of increasing the fertility of a male by administering a fertility associated antigen to the male. Preferably, the male is a human or bovine male.

Thus, another embodiment of the present invention is methods of producing a fertility associated antigen.

Other preferred embodiments of the present invention are methods of assaying the fertility of a mammal comprising detecting the presence or absence of a FAA SNP in said mammal and correlating the presence or absence of a FAA SNP with the fertility of said mammal. Preferably, the method comprises amplification a nucleic acid sample containing the FAA SNP and probing for the presence or absence of the FAA SNP. In another preferred embodiment, the method comprises using an antibody specific for the FAA SNP and probing a protein sample and/or sperm sample from said mammal and correlating the presence or absence of a FAA SNP with the fertility of said mammal.

Another preferred embodiment is a method of determining the reproductive fitness of a mammal comprising determining the nucleotide sequence of said FAA SNP;quantifying the fertility of more than one mammal containing said FAA SNP and correlating the frequency of said FAA SNP to the reproductive fitness of said mammals.

Other objects of the present invention are kits which contain the necessary reagents to conduct such assay methods.

### BRIEF DESCRIPTION OF THE DRAWINGS

FIG. 1. Reversed-phase high performance liquid chromatography (RP-HPLC) of bovine heparin-binding proteins from seminal fluid and sperm membranes and identification of bovine fertility-associated antigen (FAA). (a) RP-HPLC separation of heparin-binding proteins isolated from bovine seminal fluid (S.f.) or from sperm extracts (inserts; Sp). Proteins were isolated by heparin-affinity chromatography and fractionated by RP-HPLC using a gradient from 5.5% acetonitrile/01% TFA to 70% acetonitrile/TFA in 55 minutes. A representative chromatogram is shown. (b) Western blot of RP-HPLC peak eluting at 26.8 minutes. HPLC fractions were analyzed by SDS-PAGE and transferred to PVDF membranes for immunostaining with a monoclonal antibody (M1). The peak eluting at 26.8 minutes in approximately 44% acetonitrile contained a 31 kDa heparin-binding protein from both seminal fluid and sperm that was recognized by M1 and corresponded to FAA. The position of molecular weight standards is shown at the right of the Figure.

FIG. 2. Partial cDNA sequences of a novel human gene (SEQ ID NOS: 2, 3, 4, and 5) isolated from prostate CDNA clones compared to the homologus CDNA sequence of bovine fertility-associated antigen gene(SEQ ID NO: 1).

FIG. 3. Deduced Partial Peptides of the human Prostate cDNA sequence (SEQ ID NOS: 7, 8, 9, and 10) compared to its homologous peptide sequence deduced from the cDNA sequence of the bovine-fertility-associated antigen (SEQ ID NO: 6) gene. The homology between the deduced partial peptides of the human gene sequences and the bovine fertility-associated antigen peptide sequence ranged from 95% to 99%. The homologies among the human peptide sequences were 96% to 100%.

FIG. 4. Chou-Fasman Predictions (Chou and Fasman, 1978) of the HC1, HC2, HC3, and HC4 peptides, demonstrating potential structure-function alterations.

FIG. 5. cDNA clone sequences from single bull sample vs pooled human samples indicated existence of genetic variation within the FAA gene.

FIG. 6. Aligrunents of Human FAA sequences of clones against human genome chromosome 3 draft sequence (SEQ ID NO: 11).

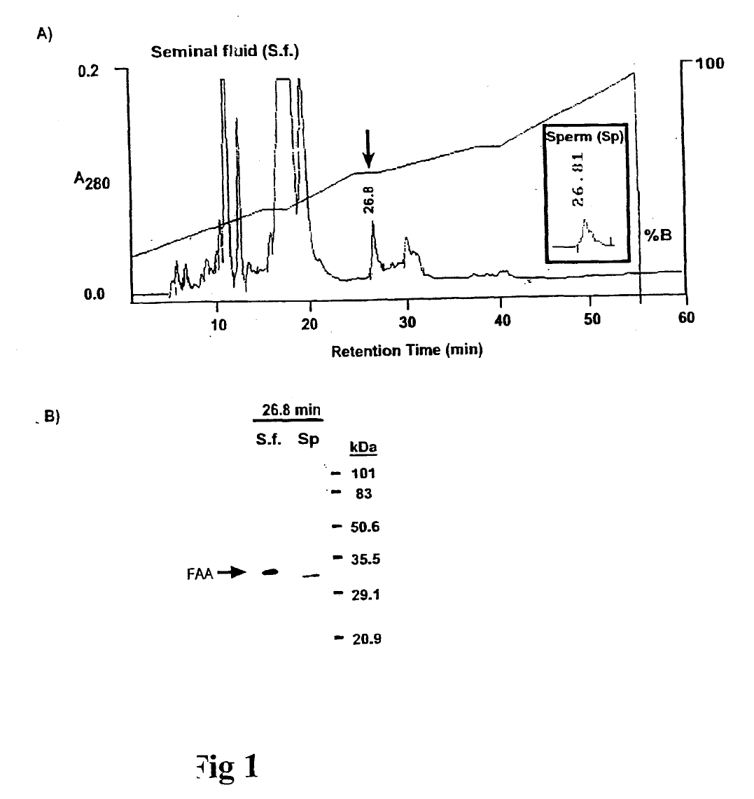

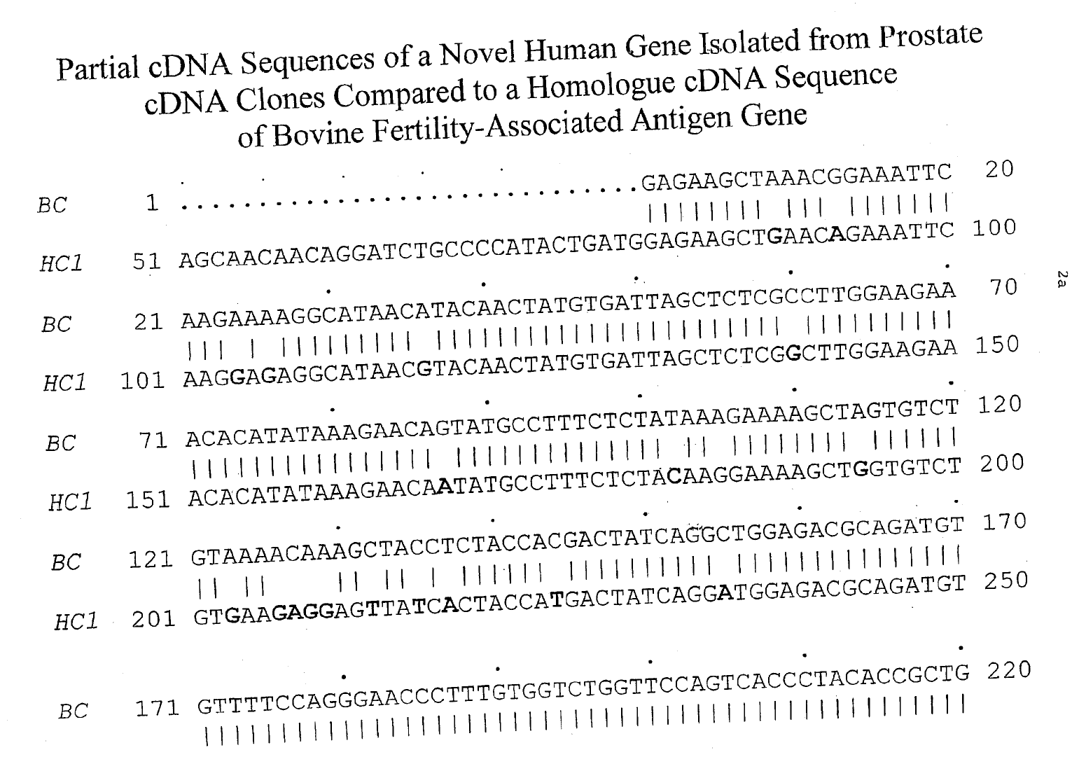

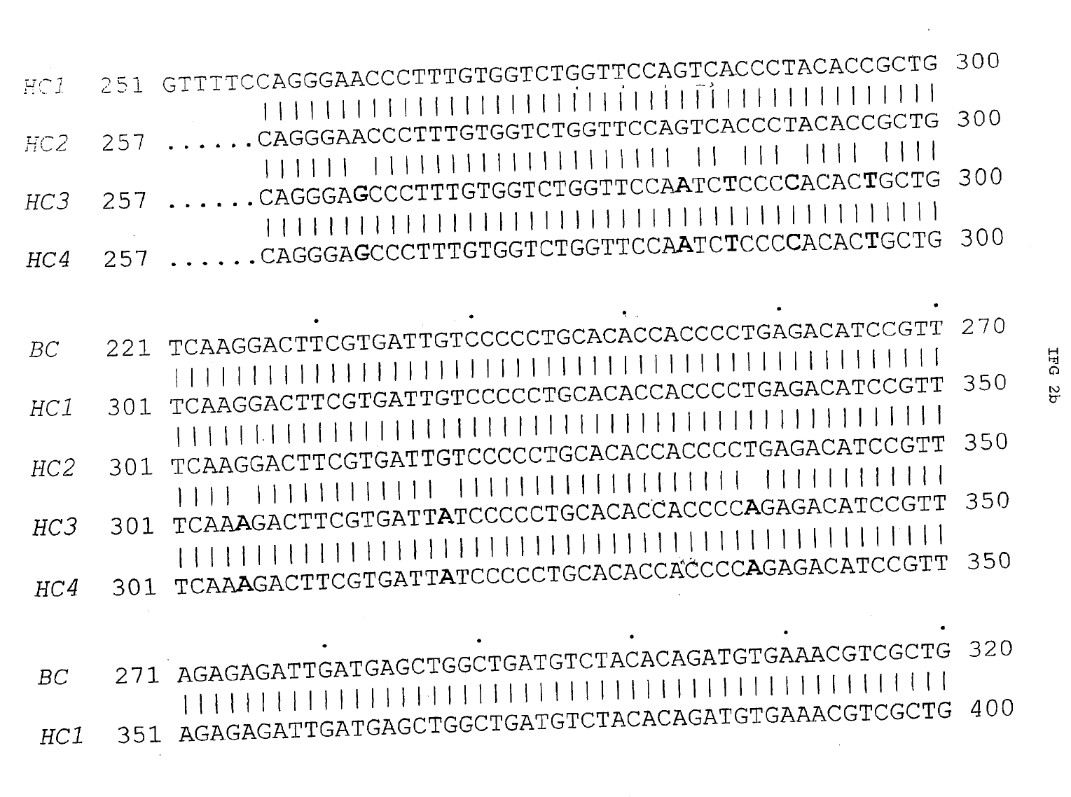

### DETAILED DESCRIPTION OF THE INVENTION

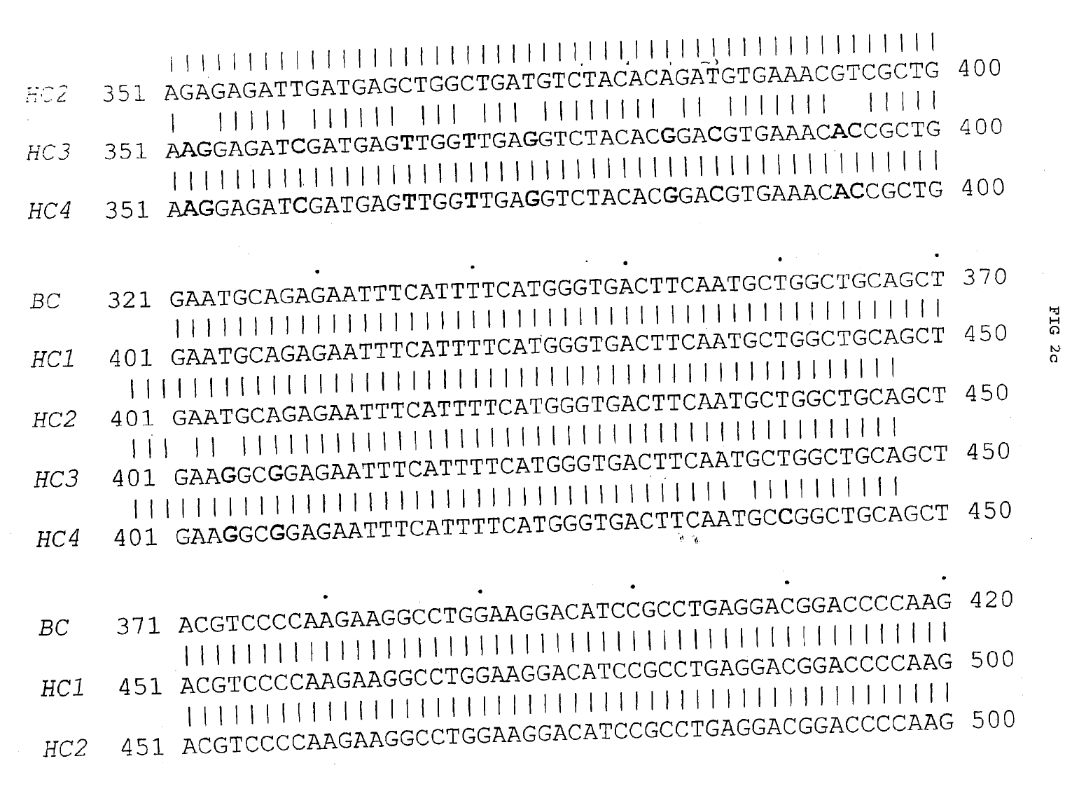

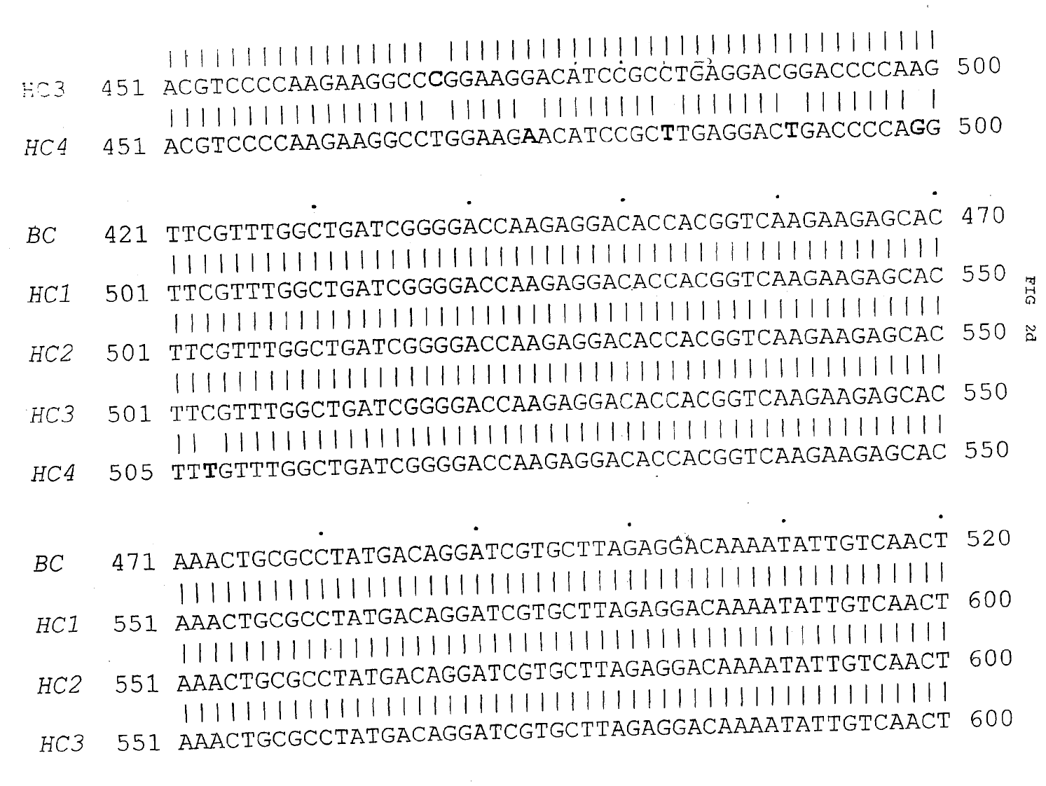

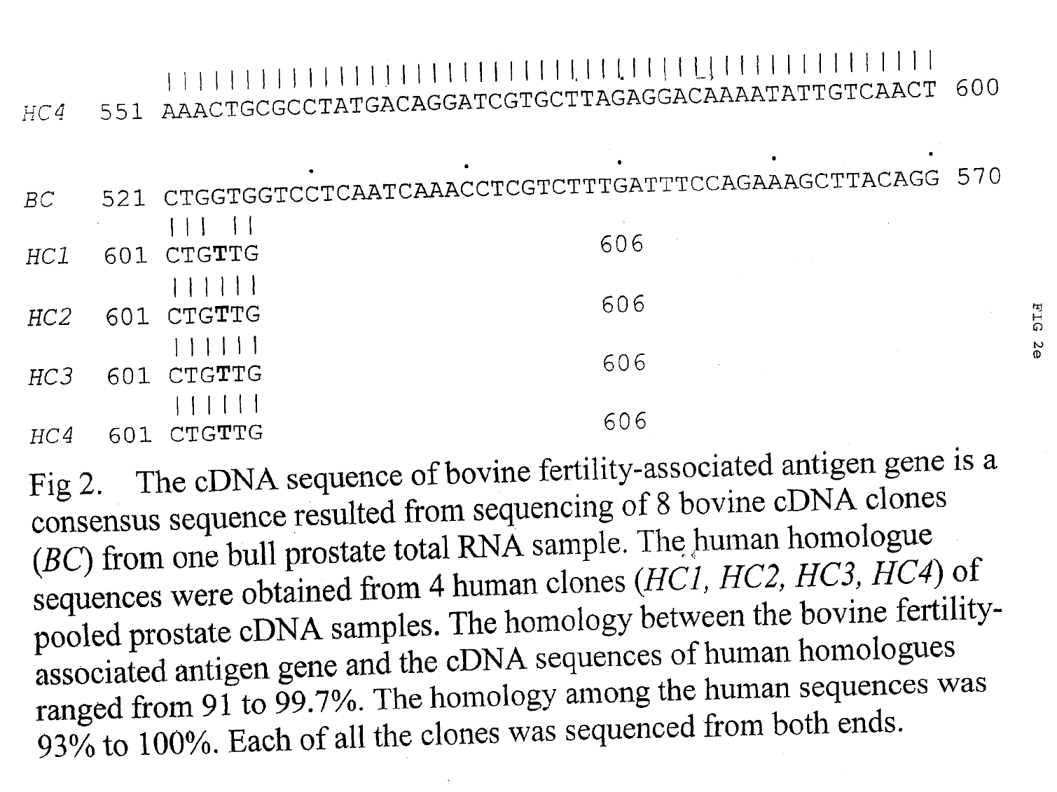

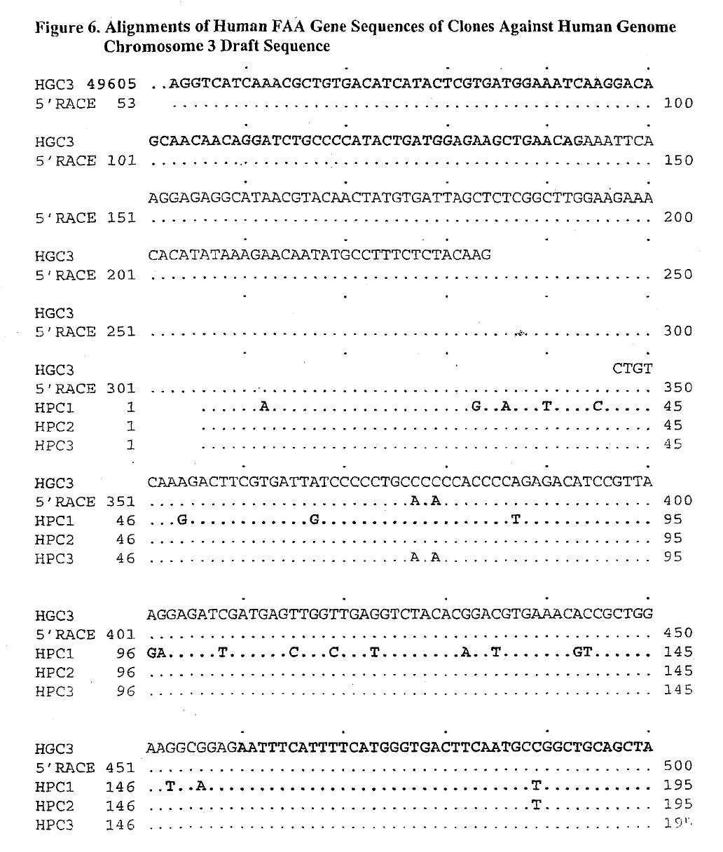

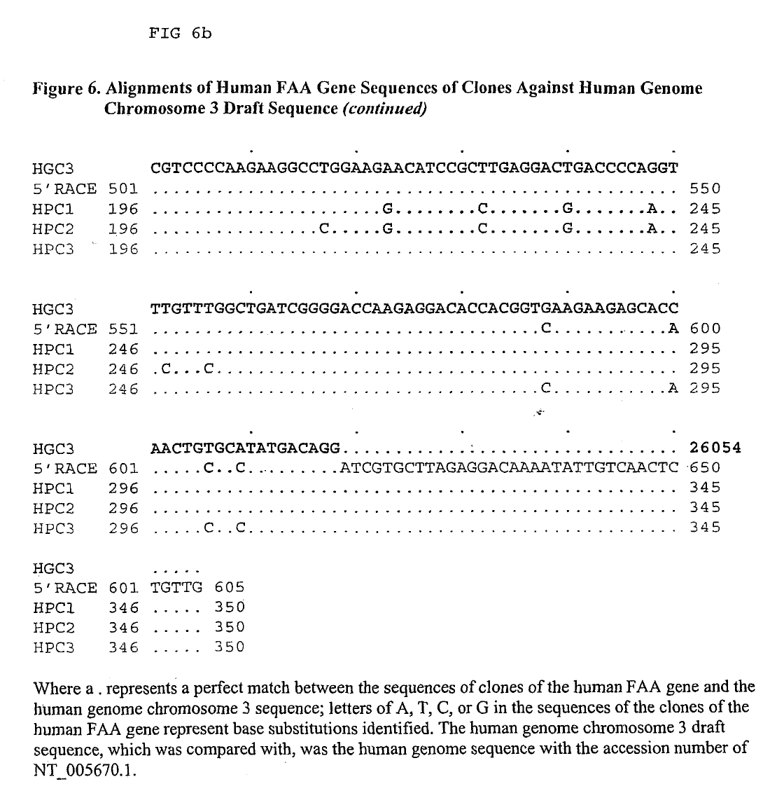

Sequences for human and bovine FAA are depicted in FIG. 2. SNPs of human FAA clones are shown in FIG. 6 and summarized in Table 3.

TABLE 3
Base Substitutions and Positions of the hFAA Gene SNPs in SEQ ID NO: 11

| | Positions | | | Sequences | | | | |
|---|---|---|---|---|---|---|---|---|
| SNP | HGC3 | 5'RACE | HPC | HGC3 | 5'RACE | HPC1 | HPC2 | HPC3 |
| 1 | | | 7 | | | A | | |
| 2 | | | 28 | | | G | | |
| 3 | | | 31 | | | A | | |
| 4 | | | 35 | | | T | | |
| 5 | | | 40 | | | C | | |
| 6 | 42909 | | 49 | A | | G | | |
| 7 | 42896 | | 62 | A | | G | | |
| 8 | 42886 | 377 | 72 | C | A | | | A |
| 9 | 42884 | 379 | 74 | C | A | | | A |
| 10 | 42876 | | 82 | A | | T | | |
| 11 | 42862 | | 96 | A | | G | | |
| 12 | 42861 | | 97 | G | | A | | |
| 13 | 42855 | | 103 | C | | T | | |
| 14 | 42848 | | 110 | T | | C | | |
| 15 | 42844 | | 114 | T | | C | | |
| 16 | 42840 | | 118 | G | | T | | |
| 17 | 42831 | | 127 | G | | A | | |
| 18 | 42828 | | 130 | C | | T | | |
| 19 | 42820 | | 138 | A | | G | | |
| 20 | 42819 | | 139 | C | | T | | |
| 21 | 42810 | | 148 | G | | T | | |
| 22 | 42807 | | 151 | G | | A | | |
| 23 | 26183 | | 184 | C | | T | T | |
| 24 | 26155 | | 212 | T | | | C | |
| 25 | 26149 | | 218 | A | | G | G | |
| 26 | 26140 | | 227 | T | | C | C | |
| 27 | 26132 | | 235 | T | | G | G | |
| 28 | 26124 | | 243 | G | | A | A | |
| 29 | 26120 | | 247 | T | | | C | |
| 30 | 26116 | | 251 | T | | | C | |
| 31 | 26084 | 588 | 283 | G | C | | | C |
| 32 | 26072 | 600 | 295 | C | A | | | A |
| 33 | 26066 | 606 | 301 | T | C | | | C |
| 34 | 26063 | 609 | 304 | A | C | | | C |

Note:
52 unmatched bases at 5' end, 37 bases at 3' end. 10 transversions and 24 transitions.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory, New York (1982) and Sambrook et al., Molecular Cloning: A Laboratory Manual, Cold Spring Harbor Laboratory, New York (1989) and the various references cited therein.

Presence of FAA on bull sperm corresponds to a 16-19% increase in fertility in bulls used for artificial insemination or natural service.

FAA can be purified by HPLC and elutes as a very hydrophobic peptide.

Suitable vectors for carrying the CDNA of the FAA gene include those vectors which can direct expression of the gene in bacterial, yeast, mammalian and/or insect cells as known in the art. One embodiment of the present invention is whereby the vectors contain an inducible or otherwise regulated expression system whereby the CDNA may be expressed under certain conditions and not expressed under other conditions. Examples of such vectors and suitable cells in which they can be introduced are described in Sambrook et al., Molecular Cloning: A Laboratory Manual, 2^{nd} Ed., Cold Spring Harbor Laboratory Press, 1989, Cold Spring Harbor, N.Y. and Current Protocols in Molecular Biology, Ausebel et al, eds., John Wiley and Sons, Inc., 2000, the contents of which are herein incorporated by reference. Methods of introducing the CDNA or vector containing the CDNA include calcium-mediated transfection, liposomes, electroporation, transformation and infection when the cDNA is contained in a viral vector as known in the art. These and other methods are described in Sambrook et al., Molecular Cloning: A Laboratory Manual, 2^{nd} Ed., Cold Spring Harbor Laboratory Press, 1989, Cold Spring Harbor, N.Y. and Current Protocols in Molecular Biology, Ausebel et al, eds., John Wiley and Sons, Inc., 2000.

Suitable culture conditions for the growth and/or production of the recombinant FAA are dependent on the cell type used. Examples of culture conditions for various cells is described in Sambrook et al., Molecular Cloning: A Laboratory Manual, 2^{nd} Ed., Cold Spring Harbor Laboratory Press, 1989, Cold Spring Harbor, N.Y.; Current Protocols in Molecular Biology, Ausebel et al, eds., John Wiley and Sons, Inc., 2000; and Cells: A Laboratory Manual Vols. 1-3), Spector et al, eds., Cold Spring Harbor Laboratory Press, Cold Spring Harbor, N.Y., 1988.

Methods of purifying FAA include high performance liquid chromatography (HPLC), ion-exchange chromatography, and size exclusion chromatography. These and other methods of protein purification are disclosed in Sambrook et al., Molecular Cloning: A Laboratory Manual, 2^{nd} Ed., Cold Spring Harbor Laboratory Press, 1989, Cold Spring Harbor, N.Y.; Current Protocols in Molecular Biology, Ausebel et al, eds., John Wiley and Sons, Inc., 2000 and Protein Purification, Scopes and Cantor, eds., Springer-Verlag, 1994 which is incorporated herein by reference.

The recombinant FAA obtained by expressing the polynucleotide as described above can be used to improve the integrity of sperm membranes and for increasing the capacitation of sperm derived from either fertile or infertile humans. In particular, the sperm from the infertile mammals will have little or no FAA present on the heads of sperm, potentially reducing capacitation of those sperm. By increasing capacitation of the sperm, the fertility of the male is thereby increased. The recombinant FAA can be administered alone, or in combination with heparin and/or other heparin binding proteins, such as tissue inhibitor metelloproteinases-2 (TIMP-2), or recombinant TIMP-2. The recombinant FAA treatment can comprise isolating the sperm from the marnmnal and then treating with the recombinant FAA and used for artificial insemination as well as used as a natural additive regent implemented by any innovative means during natural reproduction process to improve the likelihood of pregnancy, which include but not limited to, for instance, direct injection of the recombinant FAA into the mammal's reproductive tract in order to increase the capacitating and thus the fertility, or introduction of the FAA CDNA or recombinant DNA of the FAA gene directly into an appropriate target organ of the mammal, whereby the in vivo expression of the cDNA will provide functional FAA or recombinant FAA, which will bind to the sperm during natural mating or artificial insemination. Preferably, the mammal is a human or a cow.

The present invention further provides a method of stabilizing sperm cells by treating the sperm with FAA and/or heparin, and/or HBP, either for cold storage or other means of storing sperm that are commonly used in the art.

Since a characteristic sequence of FAA is shown, see FIG. 2, it is possible to prepare functional derivatives of FAA as well. By “functional derivative” is meant a fragment, variant, analog, agonist, or chemical derivative of FAA, which terms are defined below.

A “functional derivative” retains at least a portion of the amino acid sequence of FAA, which permits its utility in accordance with the present invention, namely, determining or affecting male fertility. A “fragment” of FAA refers to any subset of the FAA molecule, that is, a shorter peptide. The fragments of interest are those, which can be used to determine or affect male fertility.

A “variant” of FAA refers to a molecule, which is substantially similar to either the entire FAA protein or fragment thereof. Variant peptides may be covalently prepared by direct chemical synthesis of the variant peptide, or by expressions of modified clones of the human FAA gene in a genetically engineered prokaryotic or eukaryotic system, using methods well known in the art.

Alternatively, amino acid sequence variants of FAA can be prepared by mutation in the DNA, which encodes the synthesized FAA. Such variants include, for example, deletions from, or insertions or substitutions of, residues within the amino acid sequence. Any combination of deletion, insertion, and substitution may also be made to arrive at the final constructs, provided that the final construct possesses the desired activity. Obviously, the mutations that will be made in the DNA encoding the variant peptides must not alter the reading frame and preferably will not create complementary regions that could produce secondary mRNA structure.

At the molecular biotechnology level, these variants ordinarily are prepared by site-directed mutagenesis employing a variety of methods known in the art, see, for example, of nucleotides in the DNA encoding the peptide molecule, thereby producing DNA encoding the variant, and thereafter expressing the DNA in recombinant cell culture. The variants typically exhibit the same qualitative biological activity as the non-variant peptide.

An “analog” of FAA refers to a molecule, which is substantially similar to either the entire molecule or a fragment thereof. The analog may be prepared by chemical synthesis or in vivo synthesis.

A “chemical derivative” of FAA contains additional chemical moieties not normally part of the FAA amino acid sequence. Covalent modifications of the amino acid sequence are included within the scope of this invention. Such modifications may be introduced into the FAA by reacting targeted amino acid residues of the peptide with an organic derivatizing agent that is capable of reacting with selected side chains or terminal residues.

Amino terminal residues can be reacted with succinic or other carboxylic acid anhydrides. Other suitable reagents for derivatizing alpha-amino-containing residues include amidoesters such as methyl picolinimidate; pyridoxal phosphate; pyridoxal; chloroborohyride; trinitrobenzenesulfonic acid; O-methylisourea; 2,4-pentanedione; and transaminase-catalyzed reacted with glyoxylate.

Specific modifications of tyrosyl residues per se have been studied extensively, with particular interest in introducing spectral labels into tyrosyl residues by reaction with aromatic diazonium compounds or tetranitromethane. Most commonly, N-acetylimidazole and tetranitromethane are use to form O-acetyl tyrosyl species and 3-nitro derivatives, respectively.

Carboxyl side groups such as aspartyl or glutamyl are selectively modified by reaction with carbodiimides (R′N—C—N—R′) such as 1-cyclohexyl-3-[2-morpholinyl-(4-ethyl)]carbodiimide or 1-ethyl-3-(4-azonia-4,4-dimethylpentyl)carbodiimide. Furthermore, aspartyl and glutamyl residues are converted to asparaginyl and glutaminyl residues by reaction with ammonium ions.

As used herein, the term “muteins” or “variants” refers to analogs of FAA in which one or more of the amino acid residues of the natural FAA, preferably 1-10, and more preferably 1-5, residues, or even only a single residue, are replaced by different amino acid residues or are deleted, or one or more amino acid residues, such as 1-10, 1-5, or only one residue are added to the natural sequence of FAA. These muteins are prepared by known synthesis techniques and/or site-directed mutagenesis techniques, or by any other known technique suitable therefor. The substitutions are preferably conservative, see, e.g., Creighton, Proteins: Structure and Molecular Properties, W. H. Freeman & Co., San Francisco, 1983; both of which are hereby incorporated by reference in their entireties.

The types of such substitutions, which can be made in the protein or peptide molecules of the present invention, may be based on analysis of the frequencies of amino acid changes between a homologous protein of different species, such as those presented in Creighton. Based upon such analysis, conservative substitutions may be defined herein as exchanges within one of the following five groups:

| | |
|---|---|
| I. Small aliphatic, nonpolar or slightly polar residues: | Ala, Ser, Thr, Pro, Gly |
| II. Polar, negatively charged residues and their amides: | Asp, Asn, Glu, Gln |
| III. Polar, positively charged residues: | His, Arg., Lys |
| IV. Large, aliphatic nonpolar residues: | Met, Leu, Ile, Val, Cys |
| V. Large aromatic residues: | Phe, Try, Trp |

Within the foregoing groups the following five substitutions are considered “highly conservative”:

Asp/Glu
His/Arg/Lys
Phe/Tyr/Trp
Met/Leu/Ile/Val

Semi-conservative substitutions are defined to be exchanges between two of groups (I)-(V) above which are limited to supergroup (A), comprising (I), (II), and (III) above, or to supergroup (B), comprising (IV) and (V) above. Substitutions are not limited to the genetically encoded, or even the naturally occurring amino acids. When the epitope is prepared by peptide synthesis, the desired amino acid may be used directly. Alternatively, a genetically encoded amino acid may be modified by reacting it with an organic derivatizing agent that is capable of reacting with selected side chains or terminal residues.

The present invention also relates to an oligonucleotide probe comprising part of a polynucleic acid as defined above, with said probe being able to act as a hybridization probe for specific detection and/or classification into types and/or subtypes of an FAA nucleic caid containing said nucleotide sequence, with said probe being possibly labelled or attached to a solid substrate.

The term “primer” refers to a single stranded DNA oligonucleotide sequence capable of acting as a point of initiation for synthesis of a primer extension product which is complementary to the nucleic acid strand to be copied. The length and the sequence of the primer must be such that they allow to prime the synthesis of the extension products. Preferably the primer is about 5-50 nucleotides. Specific length and sequence will depend on the complexity of the required DNA or RNA targets, as well as on the conditions of primer such as temperature and ionic strength. The term “probe” refers to single stranded sequence-specific oligonucleotides which have a sequence which is complementary to the target sequence of the FAA gene(s) to be detected. Preferably, these probes are about 5 to 50 nucleotides long, more preferably from about 10 to 25 nucleotides. Preferred primers are from about 18 to 23 nucleotides in length, without internal homology or primer-primer homology. It is also desirable for the primers to form more stable duplexes with the target DNA at the primer's 5′-ends than at their 3′-ends, because this leads to less false priming. Stability can be approximated by GC content, since GC base pairs are more stable than AT pairs, or by nearest neighbor thermodynamic parameters. Breslauer et al., “Predicting DNA duplex stability from base sequence”, Proc. Nat'l Acad. Sci. USA 83: 3746-3750 (1986).

The amplification method used can be either polymerase chain reaction (PCR; Saiki et al., 1988), ligase chain reaction (LCR; Landgren et al., 1988; Wu & Wallace, 1989; Barany, 1991), nucleic acid sequence-based amplification (NASBA; Guatelli et al., 1990; Compton, 1991), transcription-based amplification system (TAS; Kwoh et al., 1989), strand displacement amplification (SDA; Duck, 1990; Walker et al., 1992) or amplification by means of Q.beta. replicase (Lizardi et al., 1988; Lomeli et al., 1989) or any other suitable method to amplify nucleic acid molecules using primer extension. During amplification, the amplified products can be conveniently labeled either using labeled primers or by incorporating labeled nucleotides. Labels may be isotopic (³²P, ³⁵S, etc.) or non-isotopic (biotin, digoxigenin, etc.). The amplification reaction is repeated between 20 and 70 times, advantageously between 25 and 45 times.

Additional factors to the selection of primers for amplification of polynucleic acids are discussed in Rylchik, W., Selection of Primers for Polymerase Chain Reaction”, in Methods in Molecular Biology, Vol. 15: PCR Protocols: Current Methods and Applications, White, B. A. ed., Humana Press, Totowa, N.J., 1993. Briefly, applying these factors, primer pairs are selected by position, similarity of melting temperature, internal stability, absence of internal homology or homology to each other, i.e., they won't stick to each other or to themselves, and the 3′-end will not form a stable hairpin loop back on itself.

To evaluate compatibility of primers for use in amplification, it is desirable to determine the predicted melting temperature for each primer. This can be accomplished in several ways. For example, the melting temperature, Tm can be calculated using either of the following equations:

Tm(° C.)=81.5+16.6×log [Na]+0.41×(% GC)−675/length

where [Na] is the concentration of sodium ions, and the % GC is in number percent, or

Tm(° C.)=2.times.(A+T)+4×(G+C)

where A, T, G, and C represent the number of adenosine, thymidine, guanosine and cytosine residues in the primer. In general, primers for coamplification should be selected to have predicted melting temperatures differing by less than 4° C.

The term “solid support” can refer to any substrate to which an oligonucleotide probe can be coupled, provided that it retains its hybridization characteristics and provided that the background level of hybridization remains low. Usually the solid substrate will be a microtiter plate, a membrane (e.g. nylon or nitrocellulose) or a microsphere (bead). Alternatively, the probes, primers and/or the nucleic acids to be detected can be immobilized on micro-chips which are then used as known in the art.

The present invention further features a kit that incorporates the components of the invention and makes possible convenient performance of the invention. Such a kit may also include other materials that would make the invention a part of other procedures, and may also be adaptable for multi-well technology.

The present invention also relates to a diagnostic kit used in determining the fertility of an individual or individuals, said kit comprising a primer and/or a probe as described above. In one embodiment of the invention, the probe and/or primer is/are attached to a solid support as described above. In another embodiment, the immobilized probes/primers can be arranged to specific locations, e.g., parallel configurations, to ease the detection and analysis. The oligonucleotide primer/probe may be a variety of natural and synthetic compositions such as synthetic oligonucleotides, restriction fragments, cDNAs, synthetic PNAs, and the like. The assay may also employ labeled oligonucleotides to allow ease of identification in the assays. Examples of labels which may be employed include radiolabels, enzymes, fluorescent compounds, streptavidin, avidin, biotin, magnetic moieties, metal binding moieties, antigen or antibody moieties, and the like.

The kit may also include DNA sampling means. The DNA sampling means is any means known to those skilled in the art. The kit may also comprise a DNA purification means such as a device or reagent for effecting cell lysis with SDS followed by proteinase K digestion, reagents such as 10× reaction buffers, thermostable polymerase, dNTPs, and the like. The DNA is then isolated from the specimen and target sequences amplified using an amplification technique. Alternatively, the DNA may be amplified or detected without isolation or purification but in the direct sample. Oligonucleotide DNA primers that target the specific polymorphic DNA region within the FAA gene are prepared so that in the PCR reaction amplification of the target sequences is achieved. This embodiment has the advantage of requiring only a small amount of sample.

The present invention also relates to a method for detecting FAA SNPs and also for assessing the fertility of a mammal comprising:

(i) possibly extracting sample nucleic acid,

(ii) amplifying the nucleic acid with at least one primer as defined above,

(iii) detecting the amplified nucleic acids.

The present invention also relates to a method for detecting FAA SNPs and also for assessing the fertility of a mammal comprising:

(i) possibly extracting sample nucleic acid,

(ii) possibly amplifying the nucleic acid with at least one primer as defined above,

(iii) hybridizing the nucleic acids of the biological sample, possibly under denatured conditions, at appropriate conditions with one or more probes as defined above, with said probes being preferably attached to a solid substrate,

(iv) possibly washing at appropriate conditions,

(v) detecting the hybrids formed.

The present invention also relates to a method for detecting the presence of one or more FAA SNPs present in a biological sample and associating the SNPs presence to a fertility phenotype, comprising:

(i) possibly extracting sample nucleic acid,

(ii) specifically amplifying the nucleic acid with at least one primer as defined above,

(iii) detecting said amplified nucleic acids.

The present invention also relates to a method for detecting the presence of one or more FAA SNPs genotypes present in a biological sample and associating the SNPs presence to a fertility phenotype, comprising:

(i) possibly extracting sample nucleic acid,

(ii) possibly amplifying the nucleic acid with at least one primer as defined above,

(iii) hybridizing the nucleic acids of the biological sample, possibly under denatured conditions, at appropriate conditions with one or more probes as defined above, with said probes being preferably attached to a solid substrate,

(iv) possibly washing at appropriate conditions,

(v) detecting the hybrids formed,

(vi) inferring the presence of one or more FAA SNPs present from the observed hybridization pattern and preferably correlating the presence or absence of one or more SNPs to a fertility phenotype.

The present invention also relates to a method as defined above, wherein said nucleic acids are labeled during or after amplification. “Label” is a substance that is detectable as it is such as radioactive substances, enzymes, fluorescence substances, etc. It also covers a substance which is capable of binding with such a detectable substance.

The expression “appropriate” hybridization and washing conditions are to be understood as stringent and are generally known in the art (e.g. Maniatis et al., Molecular Cloning: A Laboratory Manual, New York, Cold Spring Harbor Laboratory, 1982).

Suitable assay methods for purposes of the present invention to detect hybrids formed between the oligonucleotide probes and the nucleic acid sequences in a sample may comprise any of the assay formats known in the art, such as the conventional dot-blot format, sandwich hybridization or reverse hybridization. For example, the detection can be accomplished using a dot blot format, the unlabelled amplified sample being bound to a membrane, the membrane being incorporated with at least one labeled probe under suitable hybridization and wash conditions, and the presence of bound probe being monitored. An alternative and preferred method is to employ single stranded conformational polymorphism (SSCP) analysis as is known in the art (see, for example, Sambrook et al., Molecular Cloning: A Laboratory Manual, 2^{nd} Ed., Cold Spring Harbor Laboratory Press, 1989, Cold Spring Harbor, N.Y.; Current Protocols in Molecular Biology, Ausebel et al, eds., John Wiley and Sons, Inc., 2000).

An alternative method is a “reverse” dot-blot format, in which the amplified sequence contains a label. In this format, the unlabelled oligonucleotide probes are bound to a solid support and exposed to the labeled sample under appropriate stringent hybridization and subsequent washing conditions. It is to be understood that also any other assay method which relies on the formation of a hybrid between the nucleic acids of the sample and the oligonucleotide probes according to the present invention may be used.

In one embodiment of the present invention, the FAA genes can be used in a method for diagnosing the fertility and/or infertility of a mammal, preferably a human or cattle, by determining the genotype of the FAA gene and identifying whether the genotype is a fertility associated genotype or a infertility associated genotype.

According to the present invention, determining the association of a particular SNP or allele with a fertility or infertility phenotype can be performed by comparing the frequency of the SNP to the reproductive fitness or fertility thereof. A preferred embodiment is to generate antibodies directed to specific FAA SNPs proteins and/or peptides. In a preferred embodiment the antibodies are monoclonal antibodies. Methods of generating, purifying and characterizing antibodies are known in the art as described, for example, in Harlow and Lane, Antibodies. A Laboratory Manual, Cold Spring Harbor Laboratory Press, Cold Spring Harbor, N.Y. (1989).

Using the antibodies, the sperm from the mammal/mammals can be assayed immunologically by its ability to bind to one or more of the antibodies directed towards various FAA SNPs. Methods of assessing immunoreactivity are known in the art and include, for example, Western Blotting, ELISA, direct in situ detection (using radiolabeled probe, immunofluorescence, colormetric, or other known means of detection). The antibody may be directly labeled or can be detected by using the appropriate secondary and/or tertiary labeled antibody directed to the FAA antibody. These and other methods are known in the art and are described, for example, in Harlow and Lane, Antibodies. A Laboratory Manual, Cold Spring Harbor Laboratory Press, Cold Spring Harbor, N.Y. (1989).

In a preferred embodiment, each sample of sperm is incubated with at least two different FAA antibodies. Preferably, the different FAA antibodies are differentially labeled with a detectable moiety or are distinguishable immunologically to react with a different secondary antibody.

In another embodiment of the present invention, determining the association of a particular polymorphism or allele with a fertility or infertility phenotype can be performed suitably by assessing the genotype of the individual or individuals and comparing the genotypes with the fertility of each individual thereby generating a statistical survey of a number of individuals in a population. This polymorphic or allelic identity of the individual or individuals can be assessed by nucleic acid detection means as described above and include, for example, direct hybridization with or without amplification, or sequencing directly or from amplified products using one or more of the probes and/or primers as described above.

Fertility or reproductive fitness of an animal can be assessed by means known in the art. Examples of which include 1) competitive fertilization, 2) osteopontin and lipocallin-typel prostaglandin synthase, 3) platelet activating factor, and 4) ATP content of sperm.

Genetic testing (also called genetic screening or genotyping) can be defined broadly as the testing of nucleic acid of a patient in an analytical capacity to determine if a patient contains mutations (or alleles or polymorphisms) that affect fertility or are in “linkage disequilibrium” with the SNP(s) affecting fertility.

Linkage disequilibrium refers to the tendency of specific alleles to occur together more frequently than would be expected by chance. Alleles at given loci are in equilibrium if the frequency of any particular set of alleles (or haplotype) is the product of their individual population frequencies. The cause of disequilibrium is often unclear. It can be due to selection for certain allele combinations, or to a recent admixture of genetically heterogeneous populations. In addition, in the case of markers that very tightly link fertility, an association of an allele (or a group of linked alleles) with the fertility and/or FAA is expected if the polymorphism arose in the recent past, so that sufficient time has not elapsed for equilibrium to be achieved through recombination events in that small chromosomal region.

Statistical analysis to relate the frequency of a particular SNP or SNPs to reproductive fitness or fertility can be performed by known methods and include, for example, a t-test, 95% confidence interval or the like as known to one of ordinary skill in the art. For example, correlation between fertility and polymorphisms in the FAA gene can be assessed using the S.A.G.E. (Statistical Analysis for Genetic Epidemiology) software package (The S.A.G.E. Project, Case Western Reserve University, Department of Epidemiology and Biostatistics. The early detection of a predisposition to infertility presents the best opportunity for medical intervention. Early genetic identification of infertility may improve the prognosis for subsequent attempts at fertilization and may facilitate early intervention to determine whether the individual may benefit from fertility treatments to avoid costly and/or emotional problems with attempted inseminations. Such method of increasing fertility are discussed above.

### EXAMPLES

Materials and Methods

Marathon-Ready™ cDNA of pooled human prostate samples (20 Caucasian males of age 20-58 years; CLONTECH, Palo Alto, Calif., USA) were used as template in initial PCR analyses with gene-specific primers designed according to the cDNA sequence of bovine FAA gene recently obtained in our lab. The PCR conditions were 45 sec at 94° C., 45 sec at 56° C., and 1 min at 72° C. for 35 cycles. The PCR products were cloned into the pCR® 4-TOPO vector (Invitrogen, Carlsbad, Calif.). After s series of screening procedures, four clones were selected and subjected to sequence analysis from both directions of the insert.

Based on the cDNA sequences of the novel human gene derived from the inserts of the four clones described above, gene-specific and nested gene-specific primers were then designed and used in conjunction with CLONTECH's adaptor primers in 5′ RACE. The 5′ RACE product was also cloned followed by sequence analysis.

All sequencing analyses were accomplished on an Applied Biosystems 373A Automated DNA sequencer utilizing the DyeDeoxy™ terminator chemistry. All sequence data Were analyzed with the GCG software (Version 10.0, Genetics Computer Group, Madison, Wis.).

Results

cDNA sequence of the bovine FAA gene has been isolated and characterized via RT-PCR, cloning and sequencing analyses of total RNA samples obtained from fresh tissue samples of bovine sex glands. The present inventors have then isolated and characterized a human homologue of the FAA gene expressed in human prostate, which shares high homology to the bovine FAA gene.

Initial PCR analysis followed by cloning and sequencing resulted in 350 bp of cDNA of a novel human gene expressed in the prostate and shared 91 to 99% homology with the identified bovine FAA gene (FIG. 2).

Subsequent 5′ RACE analysis added 256 bp of additional cDNA sequence to the novel human gene, produced a cDNA of 606 bp in length, approximately ⅔ of the expected size of the human gene (FIG. 2). The cDNA sequence of bovine fertility-associated antigen gene is a consensus sequence resulted from sequencing of 8 bovine cDNA clones (BC) established from one bull prostate total RNA sample. The human homologue sequences were obtained from 4 human clones (HC1, HC2, HC3, and HC4) of pooled prostate cDNA samples. The homology between the bovine-fertility-associated antigen gene and the cDNA sequences of the human homologues ranged from 91 to 99.7%. The homology among the human sequences was 93% to 100%. Each of all the clones was sequenced from both ends.

Homology, said above, a concept arose from classical studies of comparative morphology, is commonly defined as the state of being homologous, referring to fundamental similarity in structure or processes between different organisms because of their having descended from a common evolutionary origin (King and Stansfield, 1985).

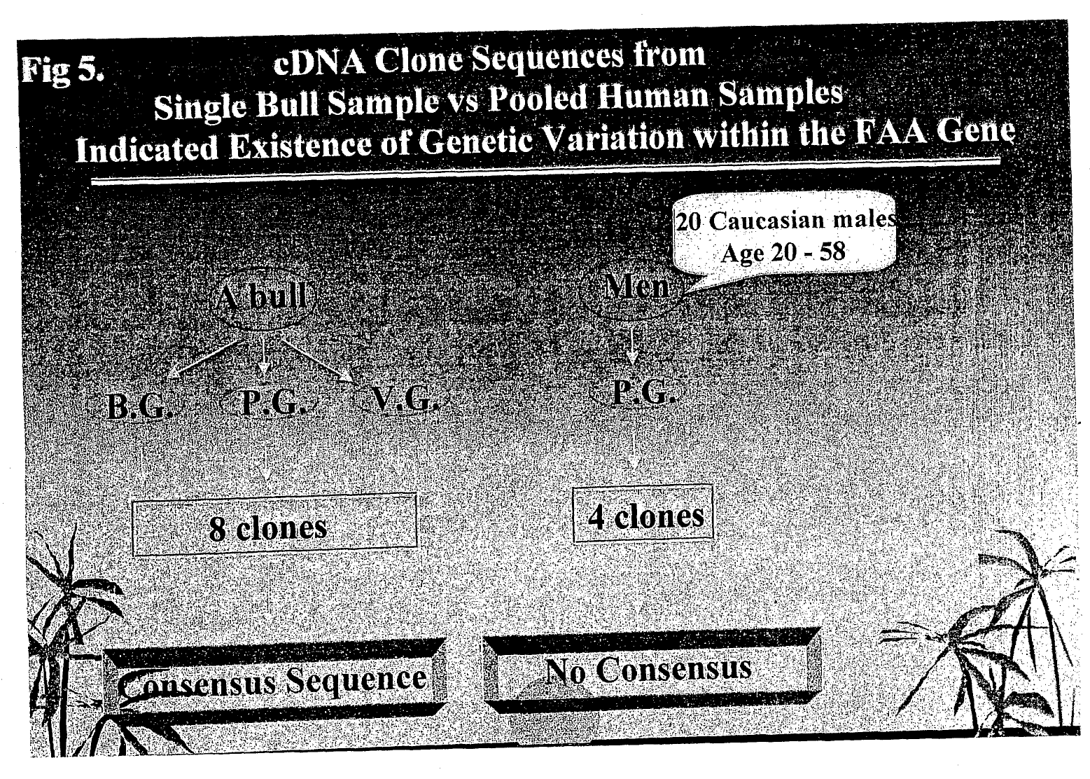

cDNA sequences of 8 selected clones, derived from total RNA samples extracted from bulbourethral gland, prostate, and seminal vesicles samples of a single bull, came to a consensus sequence. The CDNA sequences of 4 clones derived from the pooled human prostate cDNA sample offered no single consensus sequence. Multiple base substitutions were identified (FIG. 2, FIG. 5).

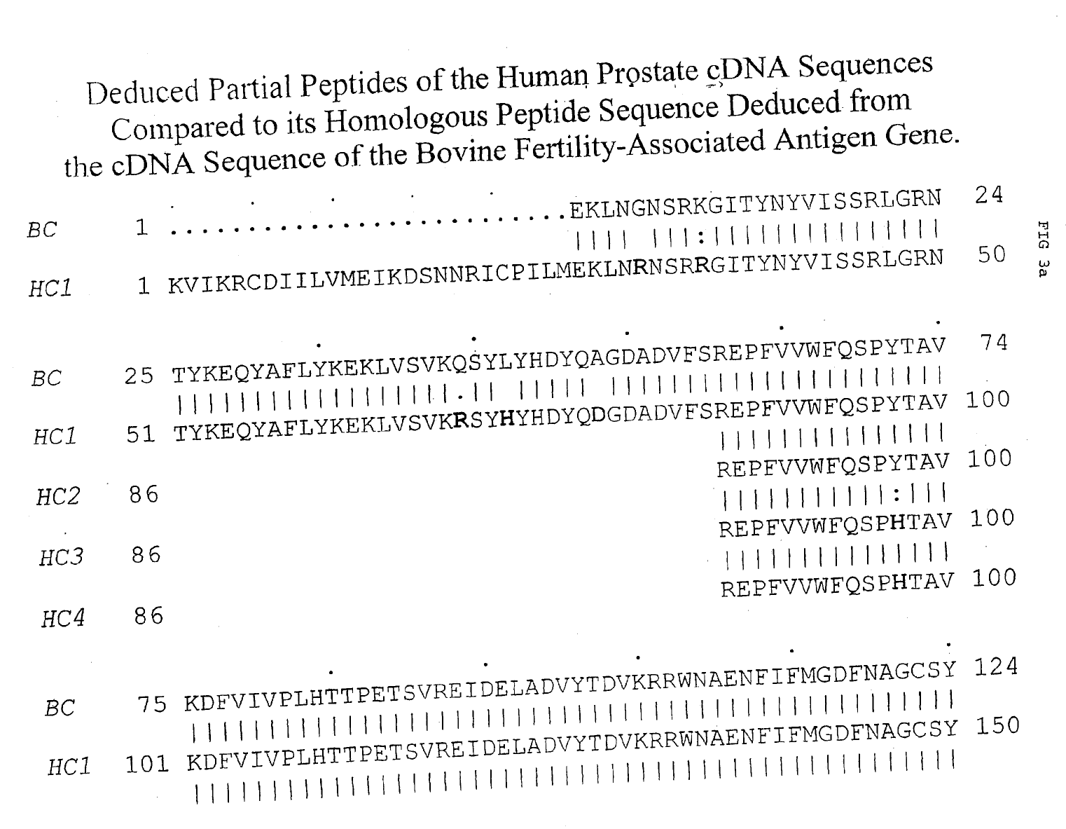

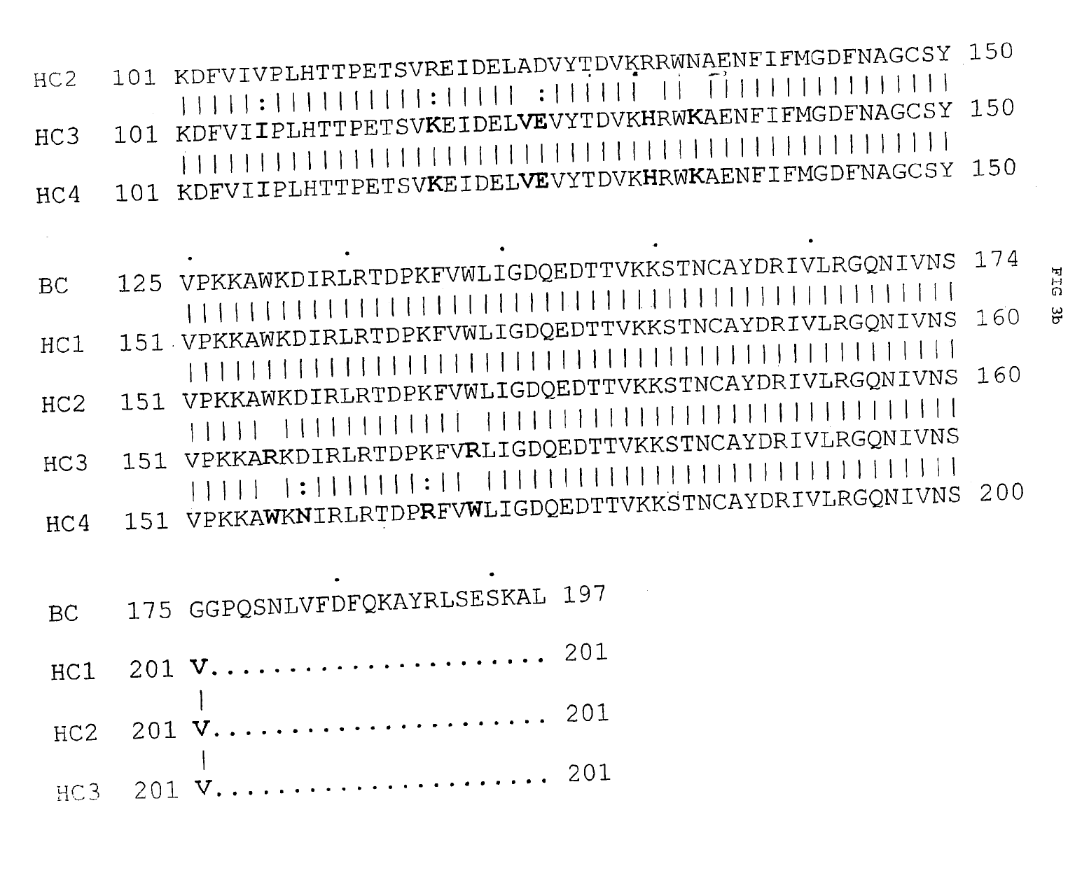

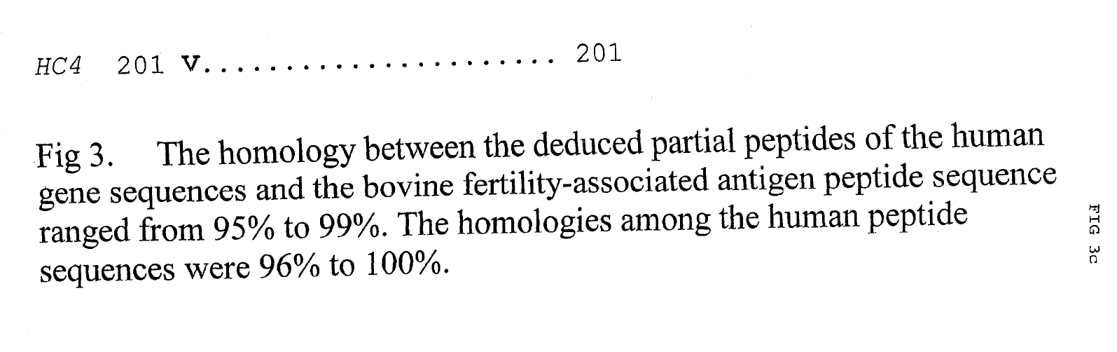

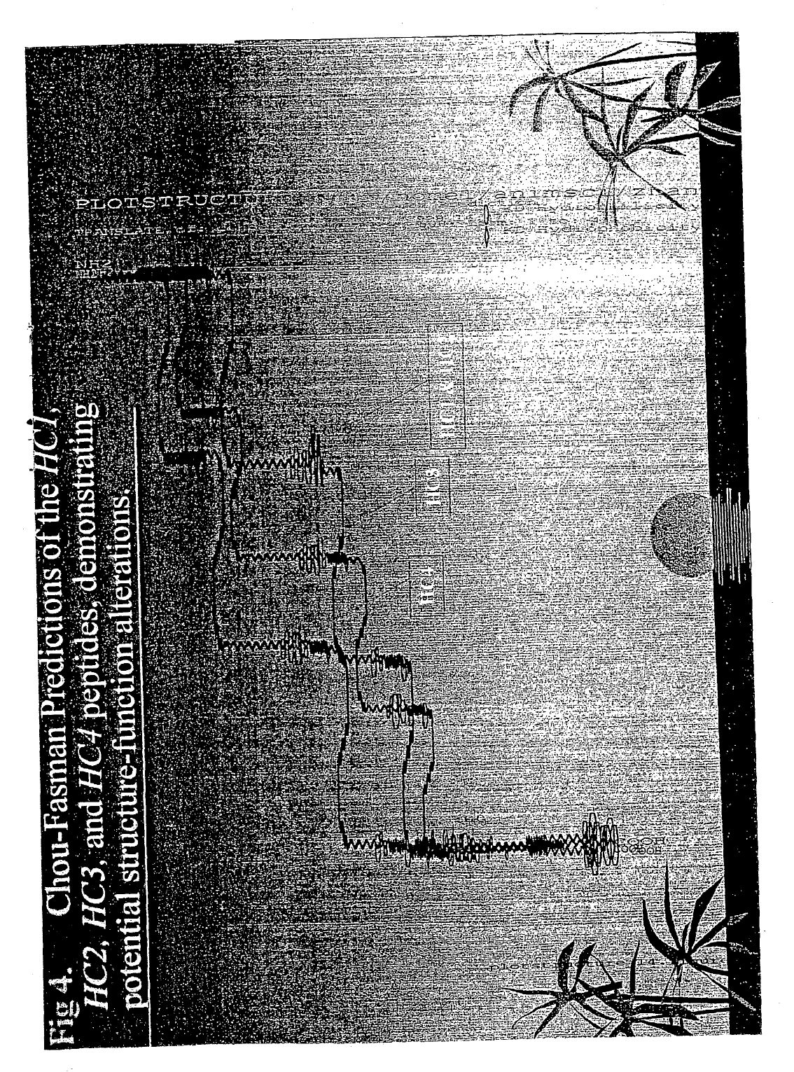

The deduced peptide sequences of the human gene shared 95% up to 99% homology with the deduced peptide of bovine FAA (FIG. 3). The homology among the deduced peptide sequences of the human gene ranged from 96% to 100%. These amino acid substitutions might introduce structural differences (FIG. 4), and led to structure-function alterations.

Of the amino acid substitutions of the deduced peptide sequences of the human FAA gene, one is in agreement with an observed amino acid substitution that was recognized when a deduced FAA sequence of one bull was compared to a known sterile bull. It is possible that this particular substitution may be indicative of male fertility characteristics. The rest of the genetic variants remain to be evaluated for physical, clinical and genetic significance.

A recombinant bovine FAA has been expressed in E. coli. Addition of the recombinant FAA to bovine sperm led to increased stability of membranes during freezing and thawing. Exposure of fresh sperm to the recombinant FAA potentiated heparin-induced capacitation in vitro.

Three expressional recombinant human FAA clones, comprising different sets of the identified SNPs of the human FAA gene, have also been established, capable of producing human recombinant FAA peptides in prokaryotic systems when induced with a chemical reagent known as IPTG. Experiments are underway in completing the cDNA sequence, constructing and assembling the genomic sequence of the human FAA gene. Future experiments will be designed to evaluate the role of various isotypes of human FAA on human sperm function, viability, and capacitation.

### REFERNCES

Bellin, M E, H E Hawkins, J N Oyarzo, R J Vanderboom, and R L Ax. 1996. Monoclonal antibody detection of heparin-binding proteins on sperm corresponding to increased fertility of bulls. J. Anim. Sci. 74:173-182.

Bellin, M E, J N Oyarzo, H E Hawkins, H M Zhang, R G Smith, D W Forrest, L R Sprott, and R L Ax, 1998. Fertility-Associated Antigen on bull sperm indicates fertility potential. J. Anim. Sci., 76(8):2032-2039.

Chou, P Y and G D Fasman, 1978. Prediction of the secondary structure of proteins from their amino acid sequence. Advances in Enzymology, 47:45-147.

Davis, B K, 1976. Inhibitory effect of synthetic phospholipid vesicles containing cholesterol on the fertilizing ability of the rabbit. Proc Soc Exp Biol Med. 152:240-243.

Florman, H M and N L First. 1988. The regulation of acrosomal exocytosis. II. The zona pellucida-induced acrosome reaction of bovine psermatozoa is controlled by extrinsic positive regulatory elements. Dev Biol. 128:464-473.

King, R C and W D Stansfield, 1985. A dictionary of genetics. Third Edition. Oxford University Press.

Lenz, R W, R L Ax, H J Grimek, and N L First. 1982. Proteiglycan from bovine follicular fluid enhances an acrosome reaction in bovine spermatozoa. Biochem Biophys Res Commun. 106:1092-1098.

Miller, D J, M A Winer, and R L Ax. 1990. Heparin-binding proteins from seminal plasma bind to bovine spermatozoa and modulate capacitation by heparin. Biol Reprod. 42:899-915.

Shivaji, S, K H Scheit, and P M Bhargava, 1990. Proteins of seminal plasma, Wiley, N.Y.

1. An isolated polynucleotide comprising the nucleic acid sequence shown in SEQ ID NO: 2, 3, 4, or 5.

2. A recombinant vector comprising the isolated polynucleotide of claim 1.

3. A host cell comprising the recombinant vector of claim 2.

4. The host cell of claim 3 , which is a bacterial cell.

5. A human fertility associated antigen encoded by the isolated polynucleotide of claim 1.

6. An isolated polypeptide comprising the amino acid sequence in SEQ ID NO: 7, 8, 9, or 10.

7. The isolated polypeptide of claim 6, which encodes a fertility-associated antigen.

8. An isolated polynucleotide which encodes the isolated polypeptide of claim 6.

9. A method of producing a fertility-associated antigen comprising
introducing the polynucleotide of claim 1 encoding FAA into a host cell;
culturing said host cell for a time and under conditions suitable for expression of the FAA; and
isolating the FAA.

10. The method of claim 9, wherein said isolating comprises purifying said FAA by chromatography.

11. The method of claim 9, wherein said host cell is a bacterial cell.

12. The method of claim 9, wherein said host cell is a mammalian cell.

13. A method of increasing the stability of a sperm cell acrosome comprising
administering FAA produced by the method of claim 9 to the sperm cell in an amount sufficient to increase the stability of said sperm cell.

14. The method of claim 13, wherein said administering is in vitro.

15. The method of claim 13, wherein said administering is in vivo.

16. A method of increasing the stability of a sperm cell acrosome comprising
administering a fertility-associated antigen comprising the amino acid sequence in SEQ ID NO: 7, 8, 9 or 10 to the sperm cell in an amount sufficient to increase the stability of said sperm cell.

17. The method of claim 16, wherein said administering is in vitro.

18. The method of claim 16, wherein said administering is in vivo.

19. A method of increasing the stability of a sperm cell acrosome comprising
administering a fertility-associated antigen encoded by the isolated polynucleotide of claim 1 to the sperm cell in an amount sufficient to increase the stability of said sperm cell.

20. The method of claim 19, wherein said administering is in vitro.

21. The method of claim 19, wherein said administering is in vivo.

22. A method of increasing the fertility of a human male comprising
(a) isolating sperm from said human male; and
(b) administering a fertility associated antigen comprising the amino acid sequence in SEQ ID NO: 7, 8, 9 or 10 in an amount sufficient to increase the fertility of said human male.

23. A method of increasing the fertility of a human male comprising
(a) isolating sperm from said human male; and
(b) administering a fertility associated antigen encoded by the isolated polynucleotide of claim 1 in an amount sufficient to increase the fertility of said human male.

24. A method of increasing the fertility of a human male comprising
(a) isolating sperm from said human male; and
(b) administering a fertility associated antigen produced by the method of claim 9 in an amount sufficient to increase the fertility of said human male.

25. An isolated polynucleotide comprising the nucleic acid sequence shown in SEQ ID NO: 1.

26. A recombinant vector comprising the isolated polynucleotide of claim 25.

27. A host cell comprising the recombinant vector of claim 26.

28. The host cell of claim 27, which is a bacterial cell.

29. A fertility associated antigen encoded by the isolated polynucleotide of claim 25.

30. An isolated polypeptide comprising the amino acid sequence in SEQ ID NO: 6.

31. The isolated polypeptide of claim 30, which encodes a fertility-associated antigen.

32. An isolated polynucleotide which encodes the isolated polypeptide of claim 30.

33. A method of producing a fertility-associated antigen comprising
introducing the polynucleotide of claim 25 encoding FAA into a host cell;
culturing said host cell for a time and under conditions suitable for expression of the FAA; and
isolating the FAA.

34. The method of claim 33, wherein said isolating comprises purifying said FAA by chromatography.

35. The method of claim 33, wherein said host cell is a bacterial cell.

36. The method of claim 33, wherein said host cell is a mammalian cell.

37. A method of increasing the stability of a sperm cell acrosome comprising
administering FAA produced by the method of claim 33 to the sperm cell in an amount sufficient to increase the stability of said sperm cell.

38. The method of claim 37, wherein said administering is in vitro.

39. The method of claim 37, wherein said administering is in vivo.

40. A method of increasing the stability of a sperm cell acrosome comprising
administering a fertility-associated antigen comprising the amino acid sequence in SEQ ID NO: 6 to the sperm cell in an amount sufficient to increase the stability of said sperm cell.

41. The method of claim 40, wherein said administering is in vitro.

42. The method of claim 40, wherein said administering is in vivo.

43. A method of increasing the stability of a sperm cell acrosome comprising
administering a fertility-associated antigen encoded by the isolated polynucleotide of claim 25 to the sperm cell in an amount sufficient to increase the stability of said sperm cell.

44. The method of claim 44, wherein said administering is in vitro.

45. The method of claim 44, wherein said administering is in vivo.

46. A method of increasing the fertility of a male mammal comprising
(c) isolating sperm from said male; and
(d) administering a fertility associated antigen comprising the amino acid sequence in SEQ ID NO: 6 in an amount sufficient to increase the fertility of said human male.

47. A method of increasing the fertility of a human male comprising
(c) isolating sperm from said human male; and
(d) administering a fertility associated antigen encoded by the isolated polynucleotide of claim 25 in an amount sufficient to increase the fertility of said human male.

48. A method of increasing the fertility of a human male comprising
(c) isolating sperm from said human male; and
(d) administering a fertility associated antigen produced by the method of claim 33 in an amount sufficient to increase the fertility of said human male.

49. A method of assaying the fertility of a mammal comprising
(a) detecting the presence or absence of a FAA SNP in said mammal; and
(b) correlating said presence or absence with the fertility of said mammal.

50. The method of claim 49, wherein the mammal is a human.

51. The method of claim 49, wherein the mammal is a bovine.

52. The method of claim 49, wherein the detecting comprises
(a) obtaining a tissue sample from said mammal,
(b) preparing from the tissue sample a nucleic acid sample; and
(c) probing the nucleic acid for the presence or absence of said FAA SNP.

53. The method of claim 52, wherein in (b) said preparing farther comprises amplifying said nucleic acid sample.

54. The method of claim 53, wherein said amplifying is by PCR.

55. The method of claim 52, wherein the FAA SNPs are selected from the group consisting of FAA SNPS 1-34 as listed in Table 3.

56. The method of claim 52, wherein the FAA is a human FAA and comprises the nucleotide sequence in SEQ ID NO: 7, 8, 9 or 10.

57. The method of claim 52, wherein the FAA is a bovine FAA and comprises the nucleotide sequence in SEQ ID NO: 1.

58. A oligonucleotide probe for assessing the fertility of a mammal which comprises at least 15 nucleotides that binds under suitably stringent conditions to a FAA SNP polynucleotide and which does not hybridize to a FAA which does not contain said SNP.

59. An assay kit comprising the oligonucleotide probe of claim 58.

60. An assay kit comprising a first oligonucleotide probe for assessing the fertility of a mammal which comprises at least 15 nucleotides that binds under suitably stringent conditions to a FAA SNP polynucleotide and which does not hybridize to a FAA which does not contain said SNP; and at least one additional oligonucleotide probe which is different from the first oligonucleotide probe and which comprises at least 15 nucleotides that binds under suitably stringent conditions to a FAA SNP polynucleotide and which does not hybridize to a FAA which does not contain said SNP including said first FAA SNP.

61. A method of detecting a FAA SNP comprising
(a) obtaining a sample which has a polynucleotide which comprises a FAA SNP;
(b) amplifying said polynucleotide with at least two oligonucleotide primers which comprise at least 15 nucleotides of the nucleotide sequence in SEQ ID NO: 2, 3, 4 or 5, wherein said oligonucleotide primers oligonucleotide primers amplify the FAA SNP; and
(c) detecting the presence or absence of said FAA SNP.

62. A method of detecting a FAA SNP comprising
(d) obtaining a sample which has a polynucleotide which comprises a FAA SNP;
(e) amplifying said polynucleotide with at least two oligonucleotide primers which comprise at least 15 nucleotides of the nucleotide sequence in SEQ ID NO: 1,
wherein said oligonucleotide primers amplify the FAA SNP; and detecting the presence or absence of said FAA SNP.

63. A method of assaying the fertility of a mammal comprising
(a) detecting the presence or absence of a FAA SNP in said mammal; and
(b) correlating said presence or absence with the fertility of said mammal.

64. The method of claim 63, wherein the mammal is a human.

65. The method of claim 63, wherein the mammal is a bovine.

66. The method of claim 63, wherein the detecting comprises incubating an antibody specific for said FAA SNP with a sperm sample from said mammal.

67. A method of determining the association of a FAA SNP with the reproductive fitness of a mammal comprising:
(a) determining the nucleotide sequence of said FAA SNP;
(b) quantifying the fertility of more than one mammal containing said FAA SNP and
(c) correlating the frequency of said FAA SNP to the reproductive fitness of said mammals.

68. The method of claim 67, wherein said mammal is a human.

69. The method of claim 67, wherein said mammal is a bovine.
